(19) United States
(12) Patent Application Publication
(10) Pub. No.: US 2018/0310875 A1
(43) Pub. Date: Nov. 1, 2018

(54) SYSTEM AND METHOD FOR DETECTING SUBSURFACE BLOOD

(71) Applicant: Covidien LP, Mansfield, MA (US)

(72) Inventors: Dwight Meglan, Westwood, MA (US); Meir Rosenberg, Newton, MA (US)

(21) Appl. No.: 15/770,087

(22) PCT Filed: Nov. 3, 2016
(86) PCT No.: PCT/US2016/060248

§ 371 Date: Apr. 20, 2018

Related U.S. Application Data
(60) Provisional application No. 62/251,203, filed on Nov. 5, 2015.

(51) Int. Cl. A61B 5/00; A61B 1/04; A61B 1/06; A61B 1/313; A61B 34/35; A61B 34/00; G06T 5/20

(52) U.S. Cl. CPC A61B 5/489; A61B 5/0086; A61B 1/04; A61B 1/0646; A61B 2090/373; A61B 34/35; A61B 34/76; G06T 5/20; A61B 2505/05; A61B 1/3132

(57) ABSTRACT

A system for detecting subsurface blood in a region of interest during a surgical procedure includes an image capture device that captures an image stream of the region of interest and a light source that illuminates the region of interest. A controller applies at least one image processing filter to the image stream, which decomposes the image stream into a plurality of color space frequency bands, generate a plurality of color filtered bands from the plurality of color space frequency bands, adds each band in the plurality of color space frequency bands to a corresponding band in the plurality of color filtered bands to generate a plurality of augmented bands, and a reconstruction filter that generates the augmented image stream from the plurality of augmented bands, which is displayed to a user during the surgical procedure.

### BACKGROUND

Minimally invasive surgeries involve the use of multiple small incisions to perform a surgical procedure instead of one larger opening or incision. The small incisions have reduced patient discomfort and improved recovery times. The small incisions have also limited the visibility of internal organs, tissue, and other matter.

Endoscopes have been used and inserted in one or more of the incisions to make it easier for clinicians to see internal organs, tissue, and other matter inside the body during surgery. These endoscopes have included a camera that is coupled to a display showing the view of organs, tissue, and matter inside the body as captured by the camera. During portions of a procedure where knowing if tissue is perfused is important, taggants such as fluorescing dyes, e.g., indocyanine green, are injected into the blood stream and then the area of interest is illuminated with powerful lasers to make the relative presence of subsurface blood visible in a camera. However, using taggants requires the use of a special type of camera to view the subsurface blood. Further, the laser sources have to be placed externally, to mitigate the heat generated, and then piped into the surgical site via optical fiber(s).

There is a need for a system that provides a clinician with a view of the subsurface blood without the need of special cameras or powerful lasers.

### SUMMARY

The present disclosure relates to minimally invasive surgery, and more specifically, image processing techniques that permit a clinician to view subsurface blood without the use of taggants or powerful lasers.

In an aspect of the present disclosure, a system for detecting subsurface blood in a region of interest during a surgical procedure is provided. The system includes an image capture device configured to be inserted into a patient and capture an image stream of the region of interest inside the patient during the surgical procedure and a light source configured to illuminate the region of interest. A controller receives the image stream and applies at least one image processing filter to the image stream to generate an augmented image stream. The image processing filter includes a color space decomposition filter configured to decompose the image into a plurality of color space frequency bands, a color filter that is configured to be applied to the plurality of color space frequency bands to generate a plurality of color filtered bands, an adder configured to add each band in the plurality of color space frequency bands to a corresponding band in the plurality of color filtered bands to generate a plurality of augmented bands, and a reconstruction filter configured to generate the augmented image stream by collapsing the plurality of augmented bands. The system also includes a display configured to display the augmented image stream to a user during the surgical procedure.

In some embodiments, the image stream includes a plurality of image frames and the controller applies the at least one image processing filter to each image frame of the image stream.

In embodiments, the color filter includes a bandpass filter, wherein a bandpass frequency of the bandpass filter corresponds to a color of interest, such as colors biased to red for arterial blood and blue-red for venous blood. The color filter isolates at least one color space frequency band from the plurality of color space frequency bands to generate the plurality of color filtered bands. The plurality of color filtered bands are amplified by an amplifier before each band in the plurality of color space frequency bands is added to the corresponding band in the plurality of color filtered bands to generate the plurality of augmented bands.

In some embodiments, the light source emits light having a wavelength between about 600 and 750 nm. In other embodiments, the light source emits light having a wavelength between about 850 and 1000 nm. In other embodiments, the light source emits visible light. In yet other embodiments, the light source emits light having a first wavelength and a second wavelength sequentially, wherein the first wavelength ranges between 600 and 750 nm and the second wavelength ranges between 850 and 1000 nm.

In another aspect of the present disclosure, a method for detecting subsurface blood in a region of interest during a surgical procedure is provided. The method includes illuminating the region of interest with a light source and capturing an image stream of the region of interest using an image capture device. The method also includes decomposing the image stream to generate a plurality of color space frequency bands, applying a color filter to the plurality of color space frequency bands to generate a plurality of color filtered bands, adding each band in the plurality of color space frequency bands to a corresponding band in the plurality of color filtered bands to generate a plurality of augmented bands, and collapsing the plurality of augmented bands to generate the augmented image stream. The augmented image stream is displayed on a display.

In embodiments, the color filter includes a bandpass filter wherein a bandpass frequency of the bandpass filter is set to a frequency that corresponds to a color of interest, such as colors biased to red for arterial blood and blue-red for venous blood. In embodiments, at least one color space frequency band is isolated from the plurality of color space frequency bands to generate the plurality of color filtered bands. The plurality of color filtered bands are amplified by an amplifier before each band in the plurality of color space frequency bands is added to the corresponding band in the plurality of color filtered bands to generate the plurality of augmented bands.

In some embodiments, the light source emits light having a wavelength between about 600 and 750 nm. In other embodiments, the light source emits light having a wavelength between about 850 and 1000 nm. In other embodiments, the light source emits visible light. In yet other embodiments, the light source emits light having a first wavelength and a second wavelength sequentially, wherein the first wavelength ranges between 600 and 750 nm and the second wavelength ranges between 850 and 1000 nm.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a block diagram of a system for augmenting an image stream of a surgical site in accordance with an embodiment of the present disclosure;

FIG. 2 is a system block diagram of the controller of FIG. 1;

FIG. 3 is a block diagram of a system for augmenting an image stream in accordance with another embodiment of the present disclosure; and

FIG. 4 is a system block diagram of a robotic surgical system in accordance with an embodiment of the present disclosure.

### DETAILED DESCRIPTION

Image data captured from an endoscope during a surgical procedure may be analyzed to detect color changes within the endoscope's field of view. Various image processing technologies may be applied to this image data to identify and amplify the causes of the color changes. For example, Eulerian image amplification techniques may be used to identify wavelength or “color” changes of light in different parts of a capture image.

Eulerian image amplification technologies may be included as part of an imaging system. These technologies may enable the imaging system to provide augmented images for a specific location within an endoscope's field of view.

One or more of these technologies may be included as part of an imaging system in a surgical robotic system to provide a clinician with additional information within an endoscope's field of view. This may enable the clinician to quickly identify, avoid, and/or correct undesirable situations and conditions during surgery.

The present disclosure is directed to systems and methods for providing augmented images in real time to a clinician during a surgical procedure. The systems and methods described herein apply image processing filters to a captured image stream to identify subsurface blood. The captured image stream is processed in real time or near real time and then displayed to the clinician as an augmented image stream. The image processing filters are applied to each frame of the captured image stream. Providing the augmented image or image stream to the clinician provides the clinician with the location of subsurface blood.

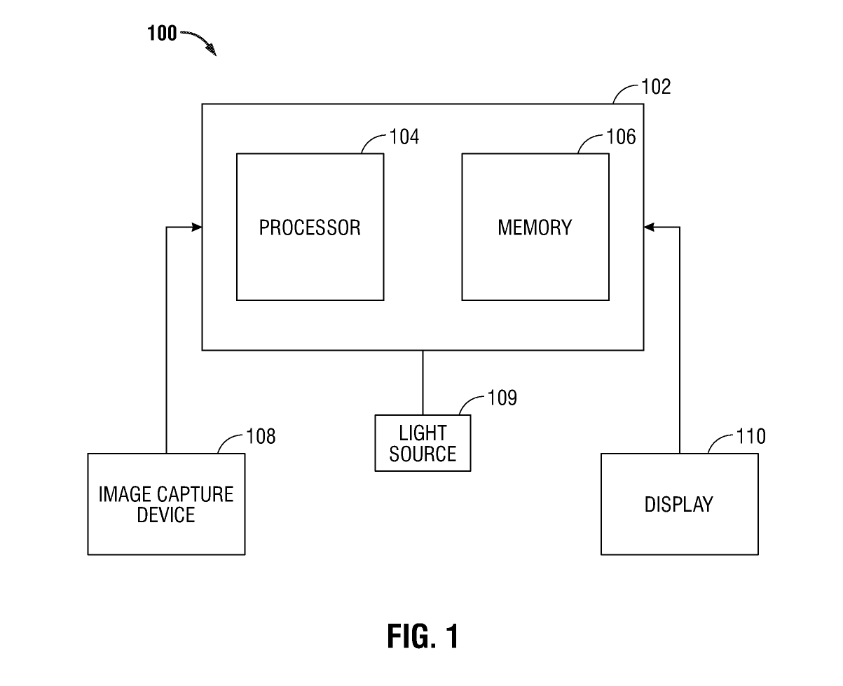

Turning to FIG. 1, a system for augmenting images and/or video of a surgical environment, according to embodiments of the present disclosure, is shown generally as 100. System 100 includes a controller 102 that has a processor 104 and a memory 106. The system 100 also includes an image capture device 108, e.g., a camera, that records an image stream. Image capture device 108 may be incorporated into an endoscope, stereo endoscope, or any other surgical tool that is used in minimally invasive surgery.

The system 100 also includes a light source 109. Light source 109, e.g., a light emitting diode (LED) or any other device capable of emitting light, may be incorporated into the image capture device 108 or it may be provided as a separate device to illuminate a surgical site. In some embodiments, light source 109 may be disposed externally of a patient and fiber optically transported to the surgical site. Light source 109 is configured to emit light at different wavelengths. For instance, light source 109 emits light sequentially with two different wavelengths, with the first wavelength ranging between about 850 to 1000 nm and the second wavelength ranging between about 600 to 750 nm. Thus, if the clinician wants to see subsurface arterial blood, light having a wavelength ranging between about 850 to 1000 nm tends to be absorbed more by arterial blood while light having a wavelength ranging between about 600 to 750 nm tends to reflect off of the arterial blood. Alternatively, if the clinician wants to see subsurface venous blood, light having a wavelength ranging between about 600 to 750 nm tends to be absorbed more by venous blood while light having a wavelength ranging between about 850 to 1000 nm tends to reflect off of the venous blood. The light source 109 may be controlled by suitable inputs on the light source 109, on the image capture device 108, or the controller 102.

A display 110, displays augmented images to a clinician during a surgical procedure. Display 110 may be a monitor, a projector, or a pair of glasses worn by the clinician. In some embodiments, the controller 102 may communicate with a central server (not shown) via a wireless or wired connection. The central server may store images of a patient or multiple patients that may be obtained using x-ray, a computed tomography scan, or magnetic resonance imaging, or the like.

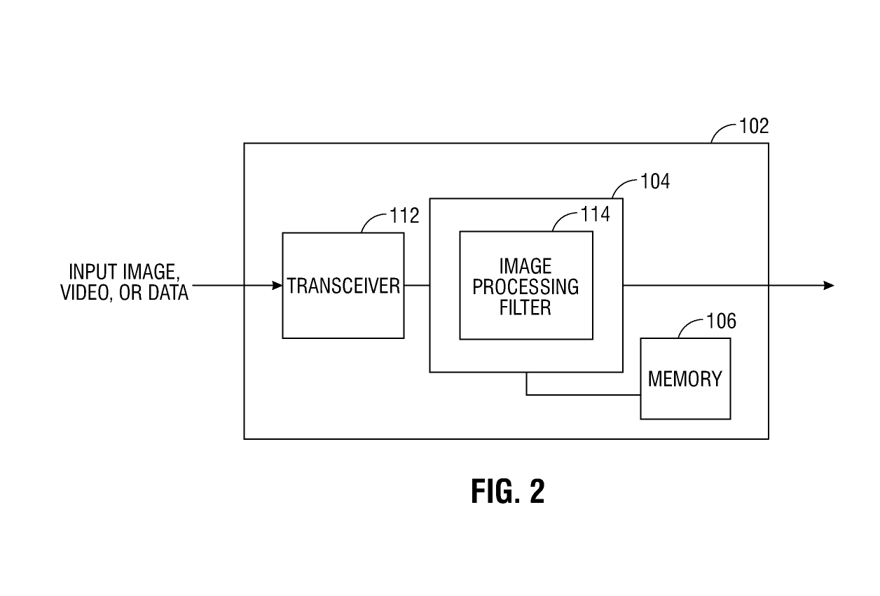

FIG. 2 depicts a system block diagram of the controller 102. As shown in FIG. 2, the controller 102 includes a transceiver 112 configured to receive still frame images or video from the image capture device 108. In some embodiments, the transceiver 112 may include an antenna to receive the still frame images, video, or data via a wireless communication protocol. The still frame images, video, or data are provided to the processor 104. The processor 104 includes an image processing filter 114 that processes the received image stream or data to generate and/or display an augmented image or image stream. The image processing filter 114 may be implemented using discrete components, software, or a combination thereof. The augmented image or image stream is provided to the display 110.

As described above, relative to venous blood, arterial blood preferentially absorbs light having a wavelength between about 850 to 1000 nm, and, relative to arterial blood, venous blood preferentially absorbs light having a wavelength between about 600 and 750 nm. Thus, when a clinician wants to see a specific type of blood, e.g., arterial or venous blood, the clinician controls the light source 109 to emit a specific wavelength. Both light wavelengths can be sequentially emitted as well to provide a differential reading that will enhance the sensitivity of the measurement of the presence of the two types of blood. The image capture device 108 captures video of the surgical site being illuminated by the selected wavelength and provides the video to the transceiver 112. In the video, arterial blood and/or venous blood will appear as whatever color is desired to highlight its presence (e.g., exaggerated red or blue for arterial and venous blood respectively).

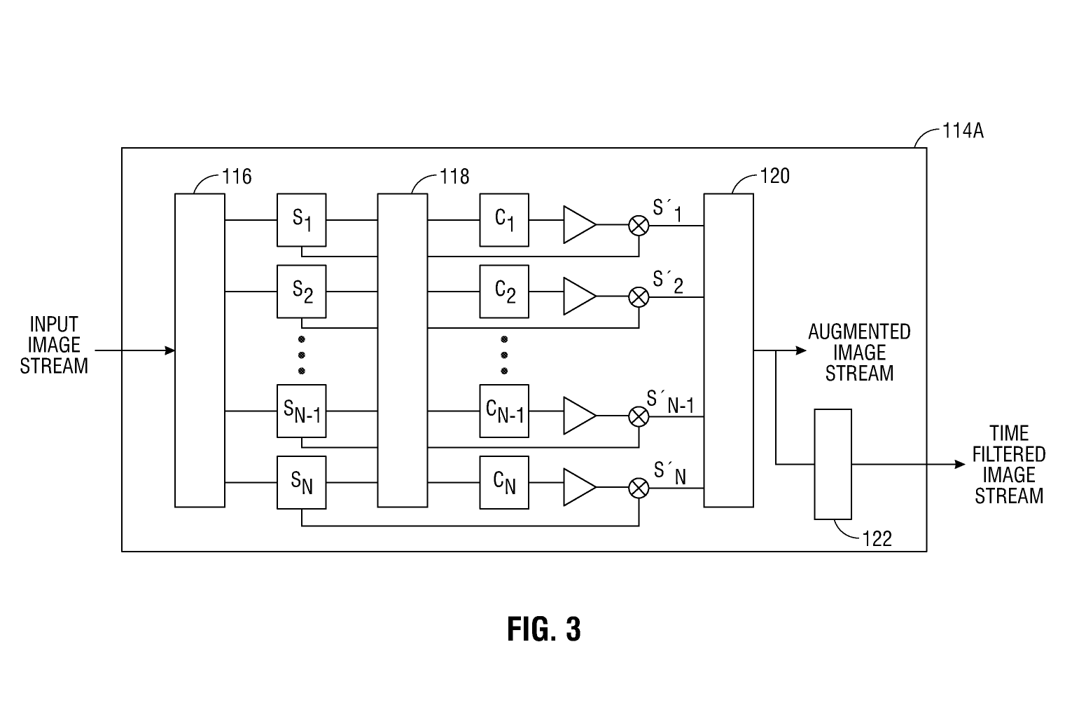

Turning to FIG. 3, a system block diagram of an image processing filter that may be applied to video received by transceiver 112 is shown as 114A. In the image processing filter 114A, each frame of a received video is decomposed into different color space frequency bands S₁ to S_{N} using a color space decomposition filter 116. The color space decomposition filter 116 uses an image processing technique known as a pyramid in which an image is subjected to repeated smoothing and subsampling.

After the frame is subjected to the color space decomposition filter 116, a color filter 118 is applied to all the color space frequency bands S₁ to S_{N} to generate color filtered bands C₁ to C_{N}. The color filter 118 is a bandpass filter that is used to extract one or more desired frequency bands. The bandpass frequency of the color filter 118 is set to a frequency range corresponding to a color, e.g., exaggerated red or blue for arterial and venous blood respectively, using a user interface (not shown). By setting the frequency range to the substantially exaggerated color typical of the type of blood vessel, the color filter 118 is capable of magnifying the visually apparent color space frequency band that corresponds to the type of blood the clinician wants to see because that type of blood will appear as the desired color in the captured images and/or video. In other words, the bandpass filter is set to a narrow range that includes the color within an acceptable tolerance and applied to all the color space frequency bands S₁ to S_{N}. Only the color space frequency band that corresponds to the set range of the bandpass filter will be isolated or passed through. All of the color filtered bands C₁ to C_{N} are individually amplified by an amplifier potentially having a unique gain “α” for each band. Because the color filter 118 isolates or passes through a desired color space frequency band, only the desired color space frequency band gets amplified. The amplified color filtered bands C₁ to C_{N} are then added to the original color space frequency bands S₁ to S_{N} to generate augmented bands S′₁ to S′_{N}. Each frame of the video is then reconstructed using a reconstruction filter 120 by collapsing augmented bands S′₁ to S′_{N} to generate an augmented frame. All the augmented frames are combined to produce the augmented image stream. The augmented image stream that is shown to the clinician includes a portion that is magnified, i.e., the portion that corresponds to the desired color space frequency band, to enable the clinician to easily identify such portion.

In some embodiments, the augmented image stream may be filtered by a time filter 122. Time filter 122 generates a baseline time varying signal, based on a pulse of the patient. The pulse may be inputted by a clinician, measured by conventional means, or determined from the image stream. The time filter 122 then averages the baseline time varying signal and removes the average signal from the augmented image stream to generate a time filtered augmented image stream. In the time filtered augmented image stream, only unique changes in blood flow are visible, thus permitting a surgeon to view situations in real time, e.g., cessation in blood flow from over clamping tissue using jaw like end effector.

The above-described embodiments may also be configured to work with robotic surgical systems and what is commonly referred to as “Telesurgery.” Such systems employ various robotic elements to assist the clinician in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

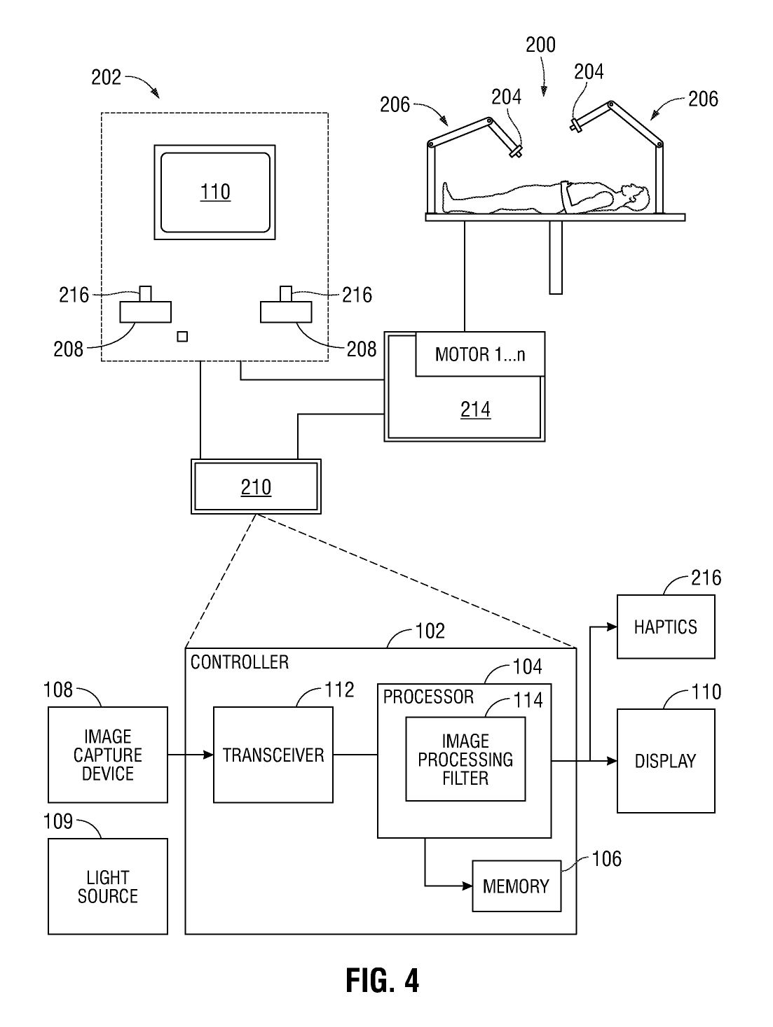

As shown in FIG. 4, a robotic surgical system 200 may be employed with one or more consoles 202 that are next to the operating theater or located in a remote location. In this instance, one team of clinicians or nurses may prep the patient for surgery and configure the robotic surgical system 200 with one or more instruments 204 while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

The robotic arms 206 of the surgical system 200 are typically coupled to a pair of master handles 208 by a controller 210. Controller 210 may be integrated with the console 202 or provided as a standalone device within the operating theater. The handles 206 can be moved by the clinician to produce a corresponding movement of the working ends of any type of surgical instrument 204 (e.g., probe, end effectors, graspers, knifes, scissors, etc.) attached to the robotic arms 206. For example, surgical instrument 204 may be a probe that includes an image capture device. The probe is inserted into a patient in order to capture an image of a region of interest inside the patient during a surgical procedure. The image processing filter 114 described above may be applied to the captured image by the controller 210 before the image is displayed to the clinician on a display 110.

The movement of the master handles 208 may be scaled so that the working ends have a corresponding movement that is different, smaller or larger, than the movement performed by the operating hands of the clinician. The scale factor or gearing ratio may be adjustable so that the operator can control the resolution of the working ends of the surgical instrument(s) 204.

During operation of the surgical system 200, the master handles 208 are operated by a clinician to produce a corresponding movement of the robotic arms 206 and/or surgical instruments 204. The master handles 208 provide a signal to the controller 210 which then provides a corresponding signal to one or more drive motors 214. The one or more drive motors 214 are coupled to the robotic arms 206 in order to move the robotic arms 206 and/or surgical instruments 204.

The master handles 208 may include various haptics 216 to provide feedback to the clinician relating to various tissue parameters or conditions, e.g., tissue resistance due to manipulation, cutting or otherwise treating, pressure by the instrument onto the tissue, tissue temperature, tissue impedance, etc. As can be appreciated, such haptics 216 provide the clinician with enhanced tactile feedback simulating actual operating conditions. The haptics 216 may include vibratory motors, electroactive polymers, piezoelectric devices, electrostatic devices, subsonic audio wave surface actuation devices, reverse-electrovibration, or any other device capable of providing a tactile feedback to a user. The master handles 208 may also include a variety of different actuators 218 for delicate tissue manipulation or treatment further enhancing the clinician's ability to mimic actual operating conditions.

The embodiments disclosed herein are examples of the disclosure and may be embodied in various forms. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in some embodiments,” or “in other embodiments,” which may each refer to one or more of the same or different embodiments in accordance with the present disclosure. A phrase in the form “A or B” means “(A), (B), or (A and B)”. A phrase in the form “at least one of A, B, or C” means “(A), (B), (C), (A and B), (A and C), (B and C), or (A, B and C)”. A clinician may refer to a surgeon or any medical professional, such as a doctor, nurse, technician, medical assistant, or the like performing a medical procedure.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, or the like. The controller may also include a memory to store data and/or algorithms to perform a series of instructions.

Any of the herein described methods, programs, algorithms or codes may be converted to, or expressed in, a programming language or computer program. A “Programming Language” and “Computer Program” includes any language used to specify instructions to a computer, and includes (but is not limited to) these languages and their derivatives: Assembler, Basic, Batch files, BCPL, C, C+, C++, Delphi, Fortran, Java, JavaScript, Machine code, operating system command languages, Pascal, Perl, PL1, scripting languages, Visual Basic, metalanguages which themselves specify programs, and all first, second, third, fourth, and fifth generation computer languages. Also included are database and other data schemas, and any other meta-languages. No distinction is made between languages which are interpreted, compiled, or use both compiled and interpreted approaches. No distinction is also made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

It should be understood that the foregoing description is only illustrative of the present disclosure. Various alternatives and modifications can be devised by those skilled in the art without departing from the disclosure. For instance, any of the augmented images described herein can be combined into a single augmented image to be displayed to a clinician. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

What is claimed is:

1. A system for detecting subsurface blood in a region of interest during a surgical procedure, the system comprising:
an image capture device configured to be inserted into a patient and capture an image stream of the region of interest inside the patient during the surgical procedure;
a light source configured to illuminate the region of interest;
a controller configured to receive the image stream and apply at least one image processing filter to the image to generate an augmented image stream, the image processing filter including:
a color space decomposition filter configured to decompose the image into a plurality of color space frequency bands;
a color filter that is configured to be applied to the plurality of color space frequency bands to generate a plurality of color filtered bands;
an adder configured to add each band in the plurality of color space frequency bands to a corresponding band in the plurality of color filtered bands to generate a plurality of augmented bands; and
a reconstruction filter configured to generate the augmented image stream by collapsing the plurality of augmented bands; and
a display configured to display the augmented image stream to a user during the surgical procedure.

2. The system of claim 1, wherein the image stream includes a plurality of image frames and the controller applies the at least one image processing filter to each image frame of the plurality of image frames.

3. The system of claim 1, wherein the color filter includes a bandpass filter.

4. The system of claim 3, wherein a bandpass frequency of the bandpass filter corresponds to a specified color.

5. The system of claim 1, wherein the color filter isolates at least one color space frequency band from the plurality of color space frequency bands to generate the plurality of color filtered bands.

6. The system of claim 1, wherein the plurality of color filtered bands are amplified by an amplifier before each band in the plurality of color space frequency bands is added to the corresponding band in the plurality of color filtered bands to generate the plurality of augmented bands.

7. The system of claim 1, wherein the light source emits light having a wavelength between about 600 and 750 nm.

8. The system of claim 1, wherein the light source emits light having a wavelength between about 850 and 1000 nm.

9. The system of claim 1, wherein the light source emits light having a first wavelength and a second wavelength sequentially, wherein the first wavelength ranges between 600 and 750 nm and the second wavelength ranges between 850 and 1000 nm.

10. A method for detecting subsurface blood in a region of interest during a surgical procedure, the method comprising:
illuminating the region of interest with a light source;
capturing an image stream of the region of interest using an image capture device;
decomposing the image stream to generate a plurality of color space frequency bands;
applying a color filter to the plurality of color space frequency bands to generate a plurality of color filtered bands;
adding each band in the plurality of color space frequency bands to a corresponding band in the plurality of color filtered bands to generate a plurality of augmented bands; and
collapsing the plurality of augmented bands to generate an augmented image stream; and
displaying the augmented image stream on a display.

11. The method of claim 9, wherein the color filter includes a bandpass filter.

12. The method of claim 11, further comprising setting a bandpass frequency of the bandpass filter, wherein the bandpass frequency corresponds to a specified color.

13. The method of claim 10, wherein applying the color filter includes isolating at least one color space frequency band from the plurality of color space frequency bands to generate the plurality of color filtered bands.

14. The method of claim 10, further comprising amplifying the plurality of color filtered bands by an amplifier before each band in the plurality of color space frequency bands is added to the corresponding band in the plurality of color filtered bands to generate the plurality of augmented bands.

15. The method of claim 10, wherein illuminating the region of interest includes emitting light having a wavelength between about 600 and 750 nm.

16. The method of claim 10, wherein illuminating the region of interest includes emitting light having a wavelength between about 850 and 1000 nm.

17. The method of claim 10, wherein illuminating the region of interest includes emitting light having a first wavelength and a second wavelength sequentially, wherein the first wavelength ranges between 600 and 750 nm and the second wavelength ranges between 850 and 1000 nm.
